(19) United States
(12) Patent Application Publication
(10) Pub. No.: US 2026/0320984 A1
(43) Pub. Date: Oct. 15, 2026

(54) TRAINING METHOD FOR MULTI-MODAL PHYSIOLOGICAL SIGNAL ESTIMATION MODEL, PHYSIOLOGICAL SIGNAL ESTIMATION METHOD AND SYSTEM

(71) Applicant: Lite-On Technology Corporation, Taipei (TW)

(72) Inventors: Jhan Yi Lin, Taipei (TW); Chiu-Ting Hsu, Taipei (TW); Kuan-Wen Chen, Taipei (TW); Ya-Ting Chan, Taipei (TW); Shih-Yu Yang, Taipei (TW); Yen-Chun Chou, Taipei (TW)

(73) Assignee: Lite-On Technology Corporation, Taipei (TW)

(21) Appl. No.: 19/533,246

(22) Filed: Feb. 8, 2026

Related U.S. Application Data
(60) Provisional application No. 63/784,999, filed on Apr. 8, 2025.

(30) Foreign Application Priority Data
Jan. 5, 2026 (TW) 115100283

(51) Int. Cl. G06V 10/774; A61B 5/00; G06V 10/143; G06V 10/44; G06V 10/74; G06V 10/776; G06V 10/80; G06V 20/59; G06V 40/16

(52) U.S. Cl. CPC G06V 10/7747; A61B 5/0035; A61B 5/0075; A61B 5/0077; G06V 10/143; G06V 10/44; G06V 10/761; G06V 10/776; G06V 10/806; A61B 2576/00; G06V 20/597; G06V 40/166; G06V 40/171

(57) ABSTRACT

A method and system for multi-modal physiological signal estimation are disclosed. The method involves receiving first and second modal image sequences corresponding to different sensing spectra. After feature extraction, an adapter module performs cross-modal feature transformation to convert original features from one modality into complementary features corresponding to the other modality, followed by optimizing an objective function.

### CROSS-REFERENCE TO RELATED APPLICATION

This application claims the priority benefit of U.S. provisional application Ser. No. 63/784,999, filed on Apr. 8, 2025 and Taiwan application serial no. 115100283, filed on Jan. 5, 2026. The entirety of each of the above-mentioned patent applications is hereby incorporated by reference herein and made a part of this specification.

### BACKGROUND

### Technical Field

The present disclosure relates to physiological signal estimation technology, and more particularly, to a training method for a multi-modal physiological signal estimation model, a physiological signal estimation method based on a single-modal source or a dual-modal source, and a physiological signal estimation system.

### Description of Related Art

Remote Photoplethysmography (rPPG) is a non-contact physiological signal measurement technology. Its principle lies in capturing subtle light absorption and reflection changes of human face skin caused by blood flow via a camera, so as to estimate physiological signals such as a heart rate of a subject. Compared to traditional Photoplethysmography (PPG) requiring sensors to directly contact skin, rPPG provides a convenient and non-invasive alternative solution, and thus has development potential in application fields such as driver monitoring and healthcare.

However, the estimation accuracy of rPPG is easily affected by external environmental factors. For visible light (RGB) cameras, the captured images are relatively sensitive to changes in ambient light. For example, in a driving scenario, a vehicle passing through tunnels, bridge shadows, or changes in sunlight conditions at different times may all lead to degradation of visible light image quality, thereby affecting the estimation accuracy of physiological signals. On the other hand, although near-infrared (NIR) cameras reduce dependence on ambient light through active lighting, their single-channel characteristic makes them relatively sensitive to movements of the subject. When the subject has head rotation or facial expression changes, NIR images are prone to generating motion artifacts, which also affects estimation results.

In the prior art, some studies attempt to combine image information of two modalities, visible light and near-infrared, hoping to improve estimation robustness through multi-modal fusion. However, simply fusing features of different modalities without considering the reliability degree of features of each modality may lead to unreliable features interfering with the overall estimation result, failing to achieve the expected improvement effect.

### SUMMARY

In view of this, the present disclosure provides a training method for a multi-modal physiological signal estimation model, a physiological signal estimation method based on a single-modal source, a physiological signal estimation method based on a dual-modal source, and a physiological signal estimation system. The present disclosure enables complementary learning between different modalities through a cross-modal feature transformation mechanism, allowing the feature extraction module of each modality to learn advantageous characteristics of the other modality during a training process. Meanwhile, the present disclosure adopts a reliability evaluation mechanism to generate a weight map so as to suppress feature contributions of unreliable regions and preserve feature information of reliable regions. Thereby, the technical solution of the present disclosure may handle single-modal or dual-modal image inputs in an inference stage, and maintain robustness of physiological signal estimation under scenarios of ambient light changes or subject movements.

One or more embodiments of the present disclosure provide a training method for a multi-modal physiological signal estimation model, comprising: receiving a first modal image sequence corresponding to a first modality, a second modal image sequence corresponding to a second modality, and a real physiological signal corresponding to the first modal image sequence and the second modal image sequence, wherein the first modality and the second modality correspond to different sensing spectra; performing feature extraction on the first modal image sequence via a first feature extraction module so as to generate a first original feature; performing feature extraction on the second modal image sequence via a second feature extraction module so as to generate a second original feature; performing cross-modal feature transformation via an adapter module, wherein the adapter module comprises a first adapter and a second adapter, the first adapter converts the second original feature into a first complementary feature, and the second adapter converts the first original feature into a second complementary feature; and adjusting parameters of the multi-modal physiological signal estimation model so as to optimize an objective function, wherein the objective function is calculated based on the real physiological signal.

One or more embodiments of the present disclosure provide a physiological signal estimation method based on a single-modal source, adapted for a multi-modal physiological signal estimation model, the multi-modal physiological signal estimation model comprising a feature extraction module, an adapter module, a reliability evaluation and feature fusion module, and a signal estimation module, the method comprising: receiving an image sequence corresponding to a first modality; performing feature extraction on the image sequence via the feature extraction module so as to generate an original feature; generating a complementary feature according to the original feature via the adapter module, wherein the complementary feature corresponds to a second modality different from the first modality, wherein the first modality and the second modality correspond to different sensing spectra; performing weighted combination on the original feature and the complementary feature via the reliability evaluation and feature fusion module so as to generate a fused feature; and estimating a physiological signal according to the fused feature via the signal estimation module.

One or more embodiments of the present disclosure provide a physiological signal estimation method based on a dual-modal source, adapted for a multi-modal physiological signal estimation model, the multi-modal physiological signal estimation model comprising a first feature extraction module, a second feature extraction module, an adapter module, a reliability evaluation and feature fusion module, and a signal estimation module, the method comprising: receiving a first modal image sequence corresponding to a first modality and a second modal image sequence corresponding to a second modality, wherein the first modality and the second modality correspond to different sensing spectra; performing feature extraction on the first modal image sequence and the second modal image sequence via the first feature extraction module and the second feature extraction module respectively so as to generate a first original feature and a second original feature; performing cross-modal feature transformation via the adapter module, wherein a first adapter converts the second original feature into a first complementary feature, and a second adapter converts the first original feature into a second complementary feature; performing intra-modal weighted combination on the first original feature and the first complementary feature via the reliability evaluation and feature fusion module so as to generate a first enhanced feature, and performing intra-modal weighted combination on the second original feature and the second complementary feature so as to generate a second enhanced feature; performing cross-modal weighted combination on the first enhanced feature and the second enhanced feature so as to generate a fused feature; and estimating a physiological signal according to the fused feature via the signal estimation module.

According to another aspect of the present disclosure, a physiological signal estimation system is provided, comprising: an image capturing device, configured to capture an image sequence of at least one modality; a processor, coupled to the image capturing device; and a storage device, coupled to the processor, configured to store a multi-modal physiological signal estimation model, wherein the model comprises a plurality of code modules, and the plurality of code modules, after being executed by the processor, cause the processor to perform the following steps: utilizing a first feature extraction module to perform feature extraction on a first modal image sequence so as to generate a first original feature; utilizing a second adapter of an adapter module to convert the first original feature into a second complementary feature, wherein the second complementary feature corresponds to a second modality different from the first modality; utilizing a reliability evaluation and feature fusion module to perform weighted combination on the first original feature and the second complementary feature so as to generate a fused feature; and utilizing a signal estimation module to estimate a physiological signal according to the fused feature.

In summary, the training method for a multi-modal physiological signal estimation model, the physiological signal estimation method, and the system provided by the present disclosure mainly perform cross-modal feature transformation via the adapter module, enabling modalities corresponding to different sensing spectra (such as visible light and near-infrared) to complementarily learn advantageous characteristics of each other in resisting ambient light interference or resisting motion artifacts, so as to compensate for innate weaknesses of a single modality. Meanwhile, by optimizing an objective function containing constraints on signal accuracy and structural consistency, it is ensured that the model effectively extracts and transforms features with physiological significance. In inference applications, the present disclosure flexibly supports single-modal or dual-modal inputs: for single-modal input, complementary features are generated to compensate for information of a missing modality, solving hardware limitations or malfunction issues; for dual-modal input, information of two modalities is integrated through cross-enhancement fusion. In conjunction with the reliability evaluation mechanism suppressing interference of unreliable regions using the weight map, the robustness and precision of physiological signal estimation are significantly improved.

It is worth emphasizing that the technical solution of the present disclosure is not merely executing mathematical operations or abstract algorithms, but rather provides a technical solution for improving performance of an image processing system itself in response to inherent physical limitations of image sensors under different environmental conditions. Specifically, visible light sensors are sensitive to ambient light changes, and near-infrared sensors are sensitive to motion of a subject, and such limitations originate from physical characteristics of different sensing spectra. The adapter module of the present disclosure learns feature mapping relationships between different sensing spectra, enabling features corresponding to a single sensing spectrum to obtain anti-interference characteristics of another sensing spectrum, thereby improving overall robustness and reliability of the image sensing system at a hardware level.

To make the aforementioned more comprehensible, several embodiments accompanied with drawings are described in detail as follows.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic diagram illustrating a physiological signal estimation system applied to a vehicle scenario according to an embodiment of the present disclosure.

FIG. 2A is a block diagram illustrating a hardware architecture of the physiological signal estimation system according to an embodiment of the present disclosure.

FIG. 2B is a block diagram illustrating a software architecture of the multi-modal physiological signal estimation model according to an embodiment of the present disclosure.

FIG. 3 is a flowchart illustrating a training method for the multi-modal physiological signal estimation model according to an embodiment of the present disclosure.

FIG. 4 is a flowchart illustrating a physiological signal estimation method based on a single-modal source according to an embodiment of the present disclosure.

FIG. 5 is a schematic diagram illustrating an architecture of the physiological signal estimation method based on a single-modal source according to an embodiment of the present disclosure.

FIG. 6 is a flowchart illustrating a physiological signal estimation method based on a dual-modal source according to an embodiment of the present disclosure.

FIG. 7 is a schematic diagram illustrating an architecture of the physiological signal estimation method based on a dual-modal source according to an embodiment of the present disclosure.

FIG. 8 is a schematic diagram illustrating a loss calculation architecture in a training stage of the multi-modal physiological signal estimation model according to an embodiment of the present disclosure.

FIG. 9A is a schematic diagram illustrating an architecture in the training stage of the multi-modal physiological signal estimation model according to an embodiment of the present disclosure.

FIG. 9B is a schematic diagram illustrating loss calculation during training of the adapter module according to an embodiment of the present disclosure.

FIG. 10 is a schematic diagram illustrating an operation of the multi-modal physiological signal estimation model based on a single-modal image sequence in an inference stage according to an embodiment of the present disclosure.

FIG. 11 is a flowchart illustrating a weight map generation mechanism according to an embodiment of the present disclosure.

### DESCRIPTION OF THE EMBODIMENTS

Reference will now be made in detail to the present embodiments of the disclosure, examples of which are illustrated in the accompanying drawings. Wherever possible, the same reference numbers are used in the drawings and the description to refer to the same or like parts.

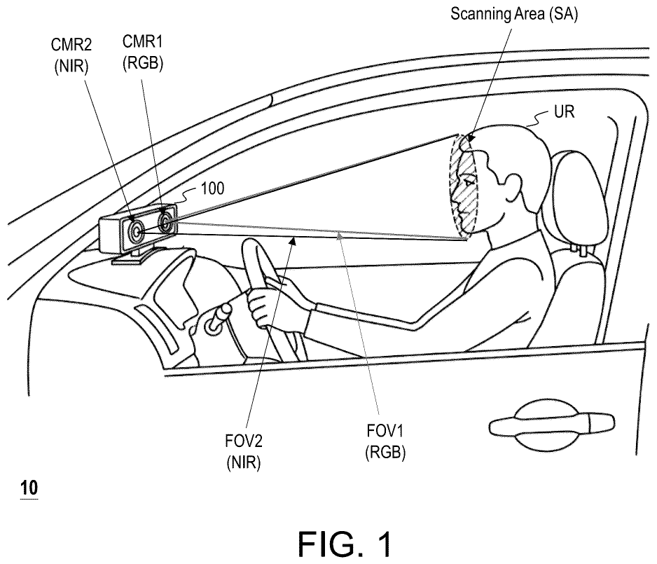

Referring to FIG. 1, in an embodiment, the physiological signal estimation system of the present disclosure is applied to a vehicle scenario. The physiological signal estimation system 10 comprises a physiological signal estimation device 100 (also referred to as an electronic device). The physiological signal estimation device 100 is disposed inside a vehicle (e.g., above a dashboard) and configured to capture a facial image of a driver UR (i.e., a subject). The physiological signal estimation system further comprises a processor (not shown). The processor is coupled to the physiological signal estimation device 100 to receive the image and perform physiological signal estimation. In this embodiment, the physiological signal estimation device 100 comprises a first camera CMR1 and a second camera CMR2.

Specifically, the first camera CMR1 is labeled as “RGB” in FIG. 1 because the first camera CMR1 is configured to sense a visible light spectrum, so as to capture a first modal image sequence corresponding to a first modality (i.e., a visible light modality). The first camera CMR1 has a first field of view FOV1 (corresponding to a field cone labeled RGB in the figure) for capturing skin color changes and subtle blood vessel pulsation information of the face of the driver UR. Conversely, the second camera CMR2 is labeled as “NIR” in FIG. 1 because the second camera CMR2 is configured to sense a near-infrared spectrum, so as to capture a second modal image sequence corresponding to a second modality (i.e., a near-infrared modality). The second camera CMR2 has a second field of view FOV2 (corresponding to a field cone labeled NIR in the figure) for providing stable structural features in an environment with low light source or drastic light changes. The first camera CMR1 and the second camera CMR2 perform image capturing toward a scanning area SA of the face of the driver UR, such that the first field of view FOV1 and the second field of view FOV2 cover the scanning area SA.

In another embodiment, the physiological signal estimation device 100 may also adopt a single-camera configuration, that is, including only the first camera CMR1 or only the second camera CMR2. When the single-camera configuration is adopted, the processor of the physiological signal estimation system executes a multi-modal physiological signal estimation model, and generates a complementary feature corresponding to another modality according to the captured single-modal image sequence via an adapter module in the model, so as to compensate for missing modal information, thereby maintaining accuracy of physiological signal estimation.

In an embodiment, in addition to being applied to the vehicle scenario, the physiological signal estimation system 10 of the present disclosure may also be extended to other scenarios requiring non-contact physiological signal monitoring, such as home healthcare, patient monitoring in medical institutions, or employee health management in office environments. In these application scenarios, an installation position and an angle of the physiological signal estimation device 100 may be adjusted according to actual environmental requirements, so as to ensure that the first field of view FOV1 or/and the second field of view FOV2 may cover effective image information of the face of the subject.

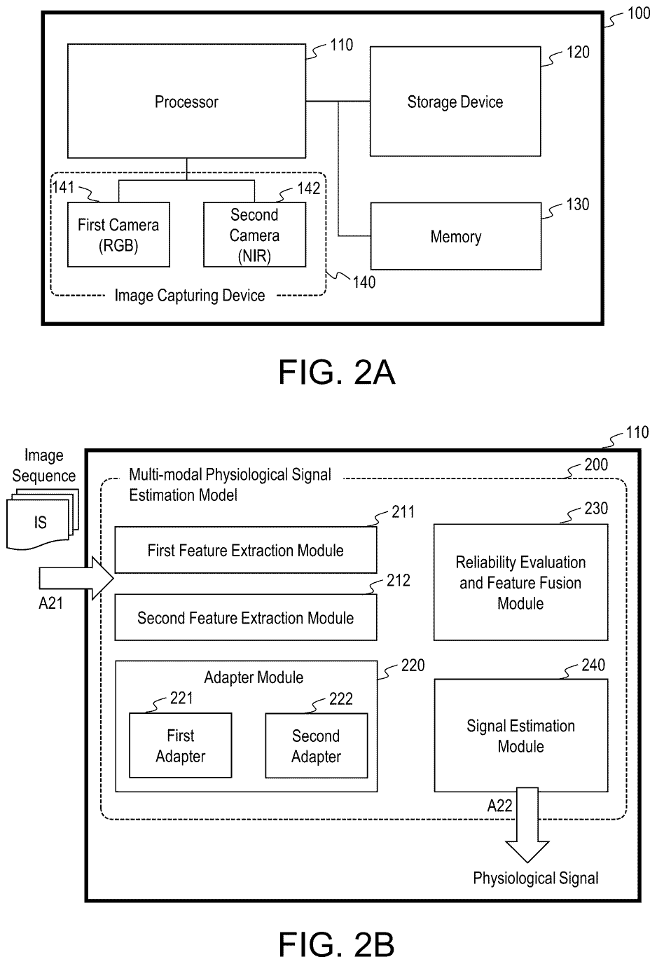

In another embodiment, the physiological signal estimation device 100 shown in FIG. 2A may also be regarded as a location of a main body of the physiological signal estimation system 10. The physiological signal estimation system 10 integrates the processor 110, the storage device 120, the memory 130, and the image capturing device 140 into an independently operating hardware unit via the physiological signal estimation device 100. The main body 100 may be implemented as an On-Board Unit, an Edge Computing Device, or an embedded system module, for being installed inside a vehicle and executing the aforementioned physiological signal estimation process.

It is worth noting that the installation position of the physiological signal estimation device 100 shown in FIG. 1 is only an exemplary illustration and is not intended to limit the scope of the present disclosure. In actual implementation, the physiological signal estimation device 100 may be disposed at other positions inside the vehicle, such as an A-pillar, a periphery of a Rearview Mirror, a dashboard assembly, or a Sun Visor. As long as an installation angle and a position of the physiological signal estimation device 100 enable its field of view (such as the first field of view FOV1 and the second field of view FOV2) to effectively cover the scanning area SA of the face of the driver UR so as to obtain a clear facial image sequence, it falls within the applicable scope of the present disclosure. In addition, in other applications, as long as the installation position of the physiological signal estimation device 100 can scan the complete face of the subject, it may fall within the applicable scope of the present disclosure.

Referring to FIG. 2A, in an embodiment, the hardware architecture of the physiological signal estimation system 100 comprises a processor 110, a storage device 120, a memory 130, and an image capturing device 140. The processor 110 is coupled to the storage device 120, the memory 130, and the image capturing device 140. The processor 110 may be a Central Processing Unit (CPU), a Graphics Processing Unit (GPU), a Neural Processing Unit (NPU), a Digital Signal Processor (DSP), an Application Processor (AP), or a combination thereof. The storage device 120 may be a non-volatile storage medium, such as a Solid State Drive (SSD), an embedded Multi-Media Card (eMMC), or a Universal Flash Storage (UFS). The memory 130 may be a volatile memory, such as a Dynamic Random Access Memory (DRAM).

The image capturing device 140 is configured to capture an image sequence of at least one modality via at least one camera. Specifically, the image capturing device 140 comprises a first camera 141 for capturing a visible light image sequence or a near-infrared image sequence. In another embodiment, the image capturing device 140 further comprises a second camera 142, such that the first camera 141 captures the visible light image sequence and the second camera 142 captures the near-infrared image sequence. In practice, the first camera 141 may comprise a lens and a Complementary Metal-Oxide-Semiconductor (CMOS) sensor for sensing a visible light band; the second camera 142 may be a monochrome CMOS sensor configured with an Infrared Filter (IR Filter) or a bandpass filter for sensing a near-infrared band (e.g., 940 nm wavelength). The first camera 141 corresponds to the first camera CMR1 in FIG. 1, and the second camera 142 corresponds to the second camera CMR2 in FIG. 1.

Referring to FIG. 2B, in an embodiment, the storage device 120 stores a multi-modal physiological signal estimation model 200. The multi-modal physiological signal estimation model 200 comprises a plurality of code modules, and the plurality of code modules are loaded into the memory 130 via the processor 110 and then executed so as to implement functions of physiological signal estimation. Specifically, the multi-modal physiological signal estimation model 200 comprises a first feature extraction module 211, a second feature extraction module 212, an adapter module 220, a reliability evaluation and feature fusion module 230, and a signal estimation module 240. These modules may be implemented by software instructions, firmware codes, or dedicated hardware circuits (such as logic gates in an ASIC or FPGA).

As shown by an arrow A21, the processor obtains an image sequence IS. The image sequence IS comprises a first modal image sequence (e.g., a visible light image sequence) or/and a second modal image sequence (e.g., a near-infrared image sequence). The first feature extraction module 211 receives the first modal image sequence and performs feature extraction so as to generate a first original feature. The second feature extraction module 212 receives the second modal image sequence and performs feature extraction so as to generate a second original feature. The first feature extraction module 211 and the second feature extraction module 212 may be implemented as Convolutional Neural Network (CNN) layers, utilizing parallel computing capabilities of the processor 110 (especially a GPU or NPU) to extract spatiotemporal features.

The adapter module 220 comprises a first adapter 221 and a second adapter 222 for performing cross-modal feature transformation. The first adapter 221 converts the second original feature into a first complementary feature so as to simulate a feature of the first modality; the second adapter 222 converts the first original feature into a second complementary feature so as to simulate a feature of the second modality. The adapter module 220 may be implemented as a neural network structure containing convolutional layers and deconvolutional layers, performing matrix operations via the processor 110 to realize transformation of a feature space. Through the operation of the adapter module 220, the present disclosure can generate complementary information of a missing modality when a single modality is input, or perform cross-feature enhancement when dual modalities are input, thereby solving the problem of accuracy degradation caused by hardware limitations or a single modality being subjected to environmental interference (such as light changes or motion artifacts).

The reliability evaluation and feature fusion module 230 is used to perform reliability evaluation on each feature and generate a weight map, and then perform weighted combination on the features according to the weight map. Specifically, the reliability evaluation and feature fusion module 230 may be implemented as codes containing an Attention Mechanism or custom weight calculation logic, calculated by the processor 110 for a variance of the feature and generating a corresponding weight matrix, and then performing an element-wise weighted operation. Thereby, the present disclosure can dynamically suppress feature contributions of unreliable regions in the image (e.g., regions obscured by shadows or shaking violently), and preserve information of reliable regions, thereby improving noise immunity and robustness of physiological signal estimation.

The signal estimation module 240 is used to estimate a physiological signal according to the fused feature. As shown by an arrow A25, the signal estimation module 240 outputs a final physiological signal (e.g., a heart rate). The signal estimation module 240 may be implemented as a decoder network or a regression model, mapping the high-dimensional fused feature back to a one-dimensional temporal signal by the processor 110. In an embodiment, the signal estimation module 240 further comprises a spectrum analysis unit (not shown) for performing Fast Fourier Transform (FFT) on the temporal signal to calculate a Power Spectral Density (PSD), and selecting a highest peak frequency as a heart rate value. This estimation method based on frequency domain analysis can effectively filter out non-periodic noise and provide more accurate heart rate readings.

In another embodiment, when the physiological signal estimation system 100 is only equipped with a camera of a single modality, the processor 110 may only execute the first feature extraction module 211 (or the second feature extraction module 212), a corresponding adapter in the adapter module 220, the reliability evaluation and feature fusion module 230, and the signal estimation module 240. For example, when only the first modal image sequence is input, the processor 110 utilizes the first feature extraction module 211 to generate the first original feature, and then utilizes the second adapter 222 to convert the first original feature into the second complementary feature. Next, the processor 110 utilizes the reliability evaluation and feature fusion module 230 to perform weighted combination on the first original feature and the second complementary feature so as to generate the fused feature, and utilizes the signal estimation module 240 to output the physiological signal.

In an embodiment, after the processor 110 estimates the physiological signal (e.g., a heart rate or heart rate variability of the driver), the processor 110 may further compare the physiological signal with a preset health threshold. If the physiological signal exceeds the preset health threshold (e.g., an excessively high heart rate indicates nervousness, or an excessively low heart rate variability indicates fatigue), the processor 110 may trigger a subsequent application procedure. The subsequent application procedure may comprise: controlling a dashboard of the vehicle to issue a warning signal, adjusting an air conditioning temperature or a music volume inside the vehicle so as to boost the spirit of the driver, or sending a health abnormality notification to a remote monitoring center via an Internet of Vehicles (IoV) system, thereby realizing intelligent driving safety monitoring.

In a further embodiment, if the processor 110 detects that the physiological signal of the driver is in a severe abnormal state (e.g., detecting cardiac arrest, extremely low blood pressure characteristics of pre-syncope, or long-term deep fatigue characteristics), the processor 110 may send an emergency intervention command to a vehicle control unit via an in-vehicle network (such as a CAN Bus). In response to the emergency intervention command, the vehicle may execute more active safety protection measures, comprising: limiting power output of the vehicle (e.g., limiting throttle response or maximum vehicle speed), activating an Advanced Driver Assistance System (ADAS) so as to take over vehicle control, turning on hazard warning lights and automatically navigating to a nearest safe shoulder to pull over, or automatically unlocking doors and notifying an emergency medical unit after the vehicle stops, so as to maximize driving safety when the driver loses consciousness or cannot control the vehicle.

In another embodiment, the physiological signal estimation system 10 of the present disclosure is further configured to execute a drunk driving prevention mechanism. The processor 110 calculates a Heart Rate Variability (HRV) index using the temporal physiological signal outputted by the signal estimation module 240, and analyzes facial skin color changes of the subject (e.g., detecting whether a flushing phenomenon caused by vasodilation occurs) in combination with the facial image captured by the first camera 141 (RGB). The processor 110 compares the HRV index and the facial skin color changes with an alcohol intake characteristic model. Since alcohol intake usually leads to a decrease in HRV (especially suppression of high-frequency components) and dilation of facial capillaries, if a comparison result matches the alcohol intake characteristic model, the processor 110 may determine that the driver is suspected to be in a drunk state. In response to this determination, the processor 110 may issue a command so as to lock an ignition system (Ignition Interlock) of the vehicle to prevent the vehicle from starting, or limit a maximum speed of the vehicle and issue a warning during driving, so as to actively prevent drunk driving accidents.

It is worth noting that although the present embodiment takes visible light (RGB) and near-infrared (NIR) as examples of the first modality and the second modality, the scope of the present disclosure is not limited thereto. In other embodiments, the image capturing device 140 may be configured to capture spectral images of other bands, such as Thermal Imaging/Long-Wave Infrared (LWIR), Short-Wave Infrared (SWIR), Ultraviolet (UV), or Hyperspectral images. As long as two selected modalities correspond to different sensing spectra and have complementary characteristics in facing environmental interference (such as light changes, smoke, occlusion) or physiological signal features (such as skin penetration depth, temperature changes), the adapter module and the feature fusion mechanism described in the present disclosure may be applied so as to improve the accuracy of physiological signal estimation.

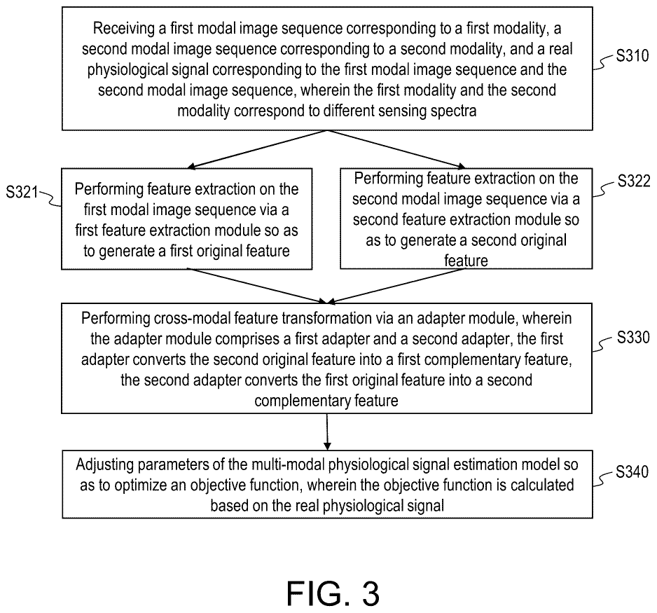

Referring to FIG. 3, in an embodiment, the training method for the multi-modal physiological signal estimation model 200 of the present disclosure comprises steps S310 to S350. The training method may be executed by a training device (e.g., the processor 110 in FIG. 2A or an external high-performance computing server).

In step S310, the training device receives a first modal image sequence IS1 corresponding to a first modality, a second modal image sequence IS2 corresponding to a second modality, and a real physiological signal corresponding to the first modal image sequence and the second modal image sequence. Wherein, the first modality and the second modality correspond to different sensing spectra (e.g., the first modality is a visible light spectrum, and the second modality is a near-infrared spectrum). The real physiological signal may be captured synchronously with the image sequences via a contact physiological sensor (such as a fingertip pulse oximeter) to serve as a supervisory label for model training.

Next, the training device executes feature extraction steps in parallel. In step S321, the training device performs feature extraction on the first modal image sequence IS1 via the first feature extraction module 211 so as to generate a first original feature RF1. In step S322, the training device performs feature extraction on the second modal image sequence IS2 via the second feature extraction module 212 so as to generate a second original feature RF2. Specifically, the first feature extraction module 211 and the second feature extraction module 212 are configured to extract spatiotemporal features containing skin blood volume change information from images of different spectra.

In step S330, the training device performs cross-modal feature transformation via an adapter module. According to this embodiment, the adapter module comprises a first adapter 221 and a second adapter 222. In this step, the first adapter 221 converts the second original feature RF2 into a first complementary feature CF1 (so as to simulate a feature of the first modality), and the second adapter 222 converts the first original feature RF1 into a second complementary feature CF2 (so as to simulate a feature of the second modality). This step aims to establish a feature mapping relationship between different modalities, enabling the model to possess an ability to reconstruct information of another modality under a single-modal input.

In step S340, the training device adjusts parameters of the multi-modal physiological signal estimation model 200 so as to optimize an objective function, wherein the objective function is calculated based on the real physiological signal. The objective function is configured to constrain performance of the model in feature extraction, cross-modal transformation, and signal estimation.

Specifically, in order to ensure that the model can simultaneously grasp accuracy of feature extraction and effectiveness of cross-modal transformation, the objective function is designed to comprise three loss terms, respectively constraining different learning objectives. First, a first loss term (L1) focuses on “signal accuracy of original features”, which is used to constrain that physiological signals respectively estimated based on the first original feature and the second original feature must remain highly similar to the real physiological signal. This ensures that the feature extraction module possesses basic feature extraction capabilities under a single modality.

Next, the second loss term (L2) focuses on “feature structural consistency”, which is used to constrain that complementary features (e.g., the first complementary feature and the second complementary feature) transformed by the adapter module must remain similar to their corresponding original features (e.g., the first original feature and the second original feature) respectively in terms of structure in a feature space. This constraint ensures that the adapter can successfully simulate a feature distribution of another modality, rather than generating meaningless noise.

Finally, the third loss term (L3) further constrains “signal accuracy of complementary features”. Even if the complementary features are obtained via transformation, the system still requires that physiological signals estimated based on these complementary features must remain consistent with the real physiological signal. This constraint strengthens semantic connotation of the complementary features, ensuring that they are not only similar in structure but also actually contain decodable physiological information. The training device jointly minimizes the above three loss terms via a backpropagation algorithm so as to update model parameters, until the model converges and forms a trained multi-modal physiological signal estimation model.

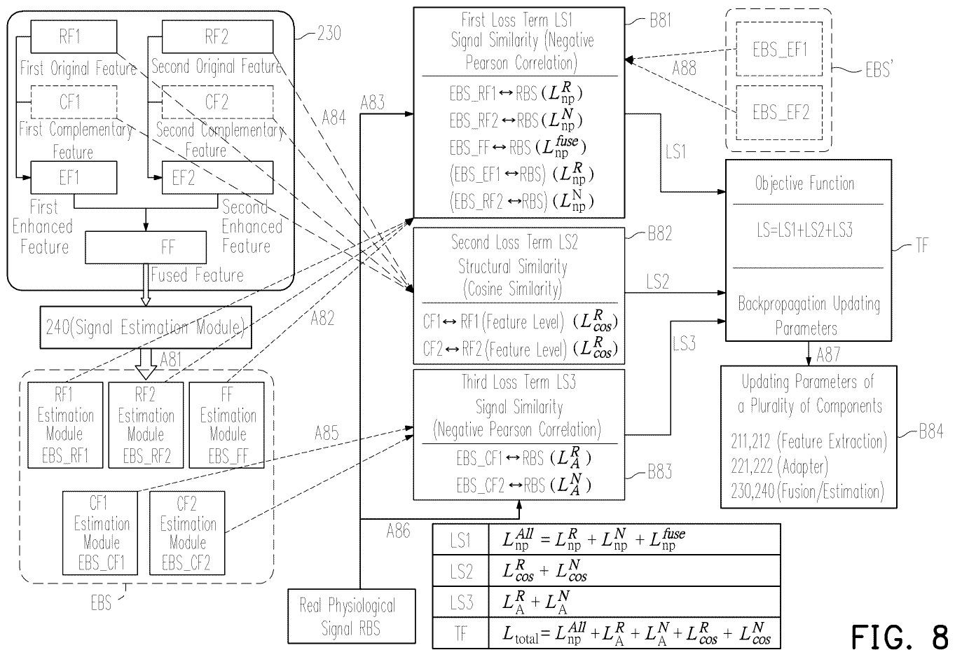

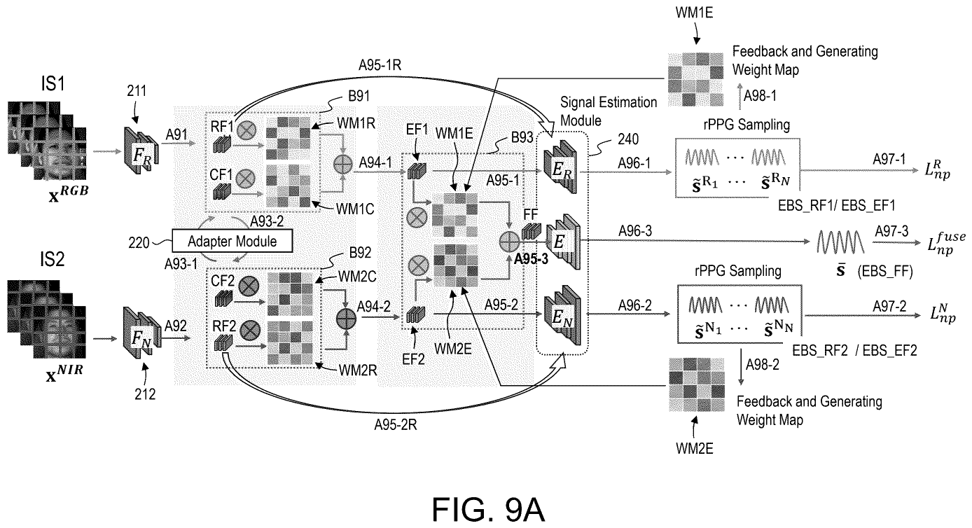

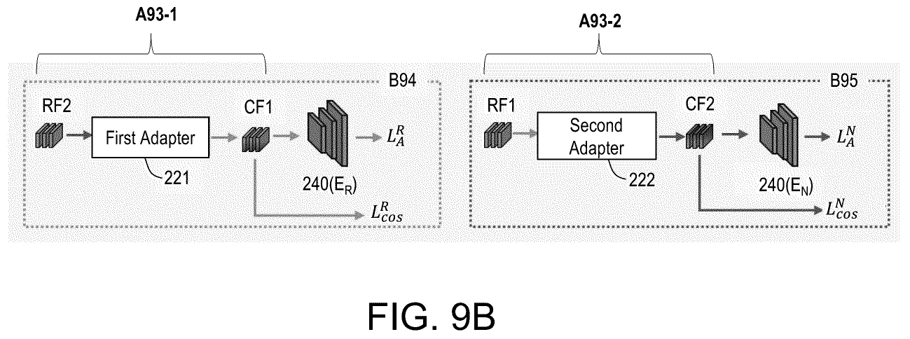

In the following, the training architecture and corresponding flow provided by the present disclosure are described in detail with reference to FIG. 8, FIG. 9A, and FIG. 9B.

First, please refer to FIG. 9A. This figure illustrates in detail a complete architecture and data flow of the multi-modal physiological signal estimation model 200 of the present disclosure in the training stage. In an embodiment, a training process starts with input of dual-modal image sequences.

As shown by an arrow A91, the first modal image sequence IS1 (corresponding to X^{RGB} in the figure) is input to the first feature extraction module 211 (labeled as F_{R}), and a first original feature RF1 is extracted after convolutional operations. As shown by an arrow A92, the second modal image sequence IS2 is input to the second feature extraction module 212 (labeled as F_{N}), and a second original feature RF2 is extracted. Next, the adapter module 220 performs cross-modal feature transformation so as to generate complementary features. As shown by an arrow A93-1, the first adapter (corresponding to an arrow path pointing upward in the adapter module 220 in the figure) receives the second original feature RF2 and converts it into a first complementary feature CF1. As shown by an arrow A93-2, the second adapter (corresponding to an arrow path pointing downward in the adapter module 220 in the figure) receives the first original feature RF1 and converts it into a second complementary feature CF2. This step ensures that the model possesses cross-modal simulation capabilities at a feature level.

To put it simply, the purpose of generating these complementary features (CF1, CF2) is to enable sensors of different modalities to serve as mutual backup. For example, visible light (RGB) images are easily interfered by environmental light flickering, but a light source of near-infrared (NIR) images is relatively stable. Converting stable NIR features into “simulated RGB features (i.e., the first complementary feature CF1)” via the adapter is equivalent to creating an “RGB feature possessing NIR anti-light interference characteristics”. In this way, when quality of a real RGB signal is poor, the system can refer to this “simulated version” so as to correct noise; even when a real RGB lens fails or is not installed, the system can also directly utilize this simulated feature so as to maintain operation, ensuring continuity and accuracy of physiological signal estimation.

Specifically, the reason why the adapter module can achieve such high-fidelity cross-modal simulation is based on a “dual constraint mechanism” applied during the training stage. First, at a structural level, the model adopts “Cosine Similarity Loss” for constraint, forcing the complementary feature generated by the adapter (e.g., a pseudo-visible light feature transformed from a near-infrared feature) to remain highly consistent with a real target modal feature in terms of spatial distribution and direction of feature vectors. Secondly, at a functional level, the model adopts “Negative Pearson Correlation Loss” for constraint, requiring that after the complementary feature is decoded by an estimator, a generated physiological signal waveform must be highly correlated with the real physiological signal (Ground Truth). Through joint optimization of these two loss functions, the adapter module (which may be implemented as a convolutional neural network) is forced to learn how to extract latent information related to physiological signals from a source modality and map it to a feature space of a target modality, thereby realizing cross-modal simulation with physiological significance.

Subsequently, the reliability evaluation and feature fusion module 230 performs two-stage feature fusion. The first stage is “intra-modal weighted combination”. As shown in a block B91, the system utilizes a first weight map WM1R and WM1C to perform element-wise weighting (⊗) on the first original feature RF1 and the first complementary feature CF1 respectively, and then adds the two (⊕) so as to generate a first enhanced feature EF1. Similarly, as shown in a block B92, the system utilizes a second weight map WM2R and WM2C to perform weighted combination on the second original feature RF2 and the second complementary feature CF2, so as to generate a second enhanced feature EF2. This stage aims to utilize complementary features so as to repair information in original features damaged by environmental interference (such as light or motion).

It should be noted that acquisition methods of these weight maps (such as WM1R, WMIC, WM2R, WM2C) mainly originate from a reliability evaluation mechanism. In an embodiment, the mechanism performs a plurality of random perturbations (e.g., adopting Dropout technique or random spatial masking) on an input feature, and calculates a statistical variance of physiological signals (e.g., heart rates) estimated under different perturbations. The variance represents prediction uncertainty of a specific spatial region; a lower variance indicates that a feature of the region is more stable and reliable, and thus is assigned a higher weight value, and vice versa, is assigned a lower weight value. In addition, these weight maps may also be upsampled via Nearest Neighbor Interpolation so as to match a spatial dimension of a feature map.

Regarding a specific implementation of generating the weight map by the reliability evaluation mechanism, in an embodiment, the mechanism adopts an evaluation strategy based on Prediction Uncertainty. Specifically, in an embodiment, the reliability evaluation mechanism comprises: dividing a feature into a plurality of spatial regions; performing random spatial masking on the plurality of spatial regions; estimating a heart rate of each region according to the masked feature; and calculating a statistical variance of the heart rates of the plurality of spatial regions, so as to generate the weight map.

More specifically, first, the system divides an input feature map (e.g., the first original feature or the second original feature) into a plurality of spatial regions in a spatial dimension. For example, the system divides a facial image or feature into a q-by-q grid. Each region independently calculates a heart rate value, which is used for judging whether the region is accurate. Next, the system performs random spatial masking for a plurality of times (e.g., K times) on these spatial regions. This operation is similar to a Dropout technique in deep learning, meaning that feature information of partial regions is randomly “turned off” or “dropped” in each operation.

For each masked feature, the signal estimation module respectively estimates a corresponding heart rate value. Since the input feature is subjected to different degrees of masking perturbation, if a certain spatial region contains a robust and stable physiological signal (e.g., a skin region of a forehead or a cheek), even if partial information is masked, the estimated heart rate value should remain consistent; conversely, if the region contains a large amount of noise (e.g., background or hair), the masking operation will cause the estimation result to fluctuate violently. Therefore, the system calculates a statistical variance of heart rate values of the spatial regions under multiple estimations. The statistical variance serves as an indicator for measuring reliability of the region: a smaller variance represents that the feature of the region is more stable, and the system assigns a higher weight value accordingly; a larger variance results in a lower weight value being assigned, finally generating a weight map reflecting importance of each region.

The second stage is “cross-modal weighted combination”. As shown in a block B93, the system further utilizes cross-modal weight maps WM1E and WM2E to weight the first enhanced feature EF1 and the second enhanced feature EF2 respectively, and fuses the two so as to generate a final fused feature FF. This two-stage fusion mechanism ensures that the final feature gathers advantages of dual modalities and filters out unreliable noise.

It is worth mentioning that generation mechanisms of the weight map WM1E and the weight map WM2E used for the second stage of cross-modal fusion are similar to the aforementioned intra-modal weight maps, with a difference lying in different objects of evaluation. Specifically, after the system generates the first enhanced feature EF1 (fused with RGB and pseudo-RGB information) and the second enhanced feature EF2 (fused with NIR and pseudo-NIR information), the reliability evaluation mechanism performs the aforementioned random spatial masking and variance calculation on these two “enhanced” features again.

The system thereby evaluates relative reliability of the “enhanced RGB feature (EF1)” and the “enhanced NIR feature (EF2)” in each spatial region. For example, if EF1 still has high variance in a certain region due to residual light interference, while EF2 performs stably in the same region, a value of the generated weight map WM2E in the region will be higher than that of WM1E. Finally, the system utilizes the two newly generated weight maps WM1E and WM2E to perform weighted fusion on EF1 and EF2, so as to generate the final fused feature FF.

The signal estimation module 240 is responsible for decoding features of each level into physiological signals. Specifically, the signal estimation module 240 comprises three estimators: (1) a first estimator E_{R}: as shown by an arrow A95-1, receiving the first enhanced feature EF1 (or as shown by an arrow A95-1R, also capable of receiving the first original feature RF1) and outputting a first estimated physiological signal EBS_EF1 (or EBS_RF1) (labeled as \tilde{S}^{R}); (2) a second estimator E_{N}: as shown by an arrow A95-2, receiving the second enhanced feature EF2 (or as shown by an arrow A95-2R, also capable of receiving the second original feature RF2) and outputting a second estimated physiological signal EBS_EF2 (or EBS_RF2) (labeled as \tilde{S}^{N}); (3) a fusion estimator E: as shown by an arrow A95-3, receiving the fused feature FF and outputting a fused estimated physiological signal EBS_FF (labeled as S).

It is worth noting that FIG. 9A also reveals a generation mechanism of the weight map. As shown by arrows A96-1 and A96-2, outputs of the estimators E_{R} and E_{N} pass through an rPPG sampling procedure (rPPG Sampling, e.g., generating a plurality of sets of predicted signals \tilde{S}₁ . . . \tilde{S}_{N} via a Dropout mechanism). The system calculates a statistical variance of these sampled signals, and as shown by arrows A98-1 and A98-2, feeds back variance information so as to generate the aforementioned weight maps (WM1E, WM2E, etc.). A region with a large variance represents that a feature there is unreliable, and thus is assigned a lower weight in the weight map. Finally, differences between each estimated physiological signal and the real physiological signal are calculated as loss terms

$${L}_{np}^{R} , {L}_{np}^{N} , {L}_{np}^{fuse} ,$$

used for jointly optimizing model parameters.

It is worth mentioning that regarding the aforementioned parameter adjustment, specifically, since the multi-modal physiological signal estimation model of the present disclosure is based on a deep learning architecture, the “parameters” mainly refer to learnable weights and biases in neural networks. Objects jointly optimized during the training process comprise: parameters of convolutional layers in the first feature extraction module and the second feature extraction module (corresponding to F_{R} and F_{N} in FIG. 9A), parameters of convolutional layers used for feature space transformation in the adapter module 220, and parameters of decoders decoding features into temporal signals in the signal estimation module (corresponding to E_{R}, E_{N} and E in FIG. 9A). Gradients of the objective function with respect to these parameters are calculated via a backpropagation algorithm, and parameter values are updated accordingly, making the model converge gradually.

In summary, the training architecture of the present disclosure enables different modalities (such as visible light and near-infrared) to perform complementary learning at a feature level via a bidirectional transformation mechanism of the adapter module. Specifically, the visible light modality can thereby learn anti-motion interference characteristics of the near-infrared modality, while the near-infrared modality can learn anti-light change characteristics of the visible light modality. In addition, combined with the weight map generated by the reliability evaluation mechanism, the model can automatically identify and suppress unreliable regions in the image (such as shadow occlusion or violent shaking), and only preserve features with high confidence for fusion. By jointly optimizing the objective function containing signal accuracy and structural consistency, this architecture can effectively train a physiological signal estimation model with high robustness, enabling it to maintain reliable estimation performance in both single-modal and dual-modal inference scenarios.

Referring to FIG. 8, FIG. 8 illustrates a loss calculation architecture of the multi-modal physiological signal estimation model 200 in a training stage. In an embodiment, the training device utilizes an objective function TF to evaluate prediction performance of the model, and updates parameters accordingly.

First, the signal estimation module 240 performs estimation according to various input features (including the first original feature RF1, the second original feature RF2, the fused feature FF, the first complementary feature CF1, and the second complementary feature CF2), as shown by an arrow A81, so as to generate a corresponding estimated physiological signal set EBS. Specifically, estimation for single-modal original features may be expressed as:

$${\tilde{s}}^{R} = {E}_{R} ( {f}^{R} ) , {\tilde{s}}^{N} = {E}_{N} ( {f}^{N} ) \qquad ( 1 )$$

wherein \tilde{s}^{R} and \tilde{s}^{N} are physiological signals estimated based on the first original feature f^{R} and the second original feature f^{N} respectively.

In order to quantify a similarity between the estimated physiological signal and the real physiological signal RBS (labeled as s), this embodiment adopts a Negative Pearson Correlation (NP) coefficient as a basic operation. For any two signal vectors Sa and Sb, a negative Pearson correlation thereof is defined as follows:

$$NP ( {s}_{a} , {s}_{b} ) = 1 - \frac{Cov ( {s}_{a} , {s}_{b} )}{\sqrt{Cov ( {s}_{a} , {s}_{a} )} \cdot \sqrt{Cov ( {s}_{b} , {s}_{b} )}} \qquad ( 2 )$$

wherein Cov(⋅) represents a covariance operation.

The first loss term LS1 (as shown in a block B81) focuses on “signal accuracy of original features and fused features”. As shown by arrows A82 and A83, the first loss term LS1 calculates a difference between the real physiological signal s and physiological signals of each modality estimated based on each feature (such as RF1, RF2, FF).

Specifically, for the first modality (such as RGB), a loss function

$${ℒ}_{np}^{R}$$

thereof is defined as an average value of negative Pearson correlations between the real signal s and estimated physiological signals

$${\tilde{s}}_{i , j}^{R}$$

of all spatial regions (S^{R}):

$${ℒ}_{np}^{R} = \frac{1}{\vert {S}^{R} \vert} {\sum }_{{\tilde{s}}_{i , j}^{R} ∈ {S}^{R}} NP ( {\tilde{s}}_{i , j}^{R} , s ) \qquad ( 3 )$$

Similarly, for the second modality (such as NIR), a loss function

$${ℒ}_{np}^{R}$$

thereof is defined as:

$${ℒ}_{np}^{N} = \frac{1}{\vert {S}^{N} \vert} {\sum }_{{\tilde{s}}_{i , j}^{N} ∈ {S}^{N}} NP ( {\tilde{s}}_{i , j}^{N} , s ) \qquad ( 4 )$$

For the fused feature FF (labeled as f̅), a loss function

$${ℒ}_{np}^{fuse}$$

corresponding to an estimated physiological signal s̅=E(f̅) thereof is defined as:

$${ℒ}_{np}^{fuse} = \frac{1}{\vert \overline{S} \vert} {\sum }_{{\overline{s}}_{i , j} ∈ \overline{S}} NP ( {\tilde{s}}_{i , j}^{N} , s ) \qquad ( 5 )$$

The first loss term LS1 is a sum of these individual losses (i.e.,

$${ℒ}_{np}^{All} = {ℒ}_{np}^{R} + {ℒ}_{np}^{N} + {ℒ}_{np}^{fuse} ) .$$

The second loss term LS2 (as shown in a block B82) focuses on “feature structural consistency”. As shown by an arrow A84, the second loss term LS2 utilizes Cosine Similarity to constrain structures of the complementary features (such as CF1, CF2) and corresponding original features (such as RF1, RF2). First, an operation of generating the complementary features by the adapter module may be expressed as:

$${\hat{f}}^{R} = {A}_{R} ( {f}^{N} ) , {\hat{f}}^{N} = {A}_{N} ( {f}^{R} ) \qquad ( 6 )$$

Next, taking the first modality as an example, a structural loss

$${ℒ}_{cos}^{R}$$

thereof is defined as follows:

$${ℒ}_{cos}^{R} = 1 - cos ( {f}^{R} , {\hat{f}}^{R} )$$

wherein f^{R} is the first original feature, \hat{f}^{R} is the first complementary feature, and cos(⋅) is a cosine similarity function. Similarly, a structural loss

$${ℒ}_{cos}^{N}$$

of the second modality is defined as:

$${ℒ}_{cos}^{N} = 1 - cos ( {f}^{N} , {\hat{f}}^{N} ) \qquad ( 7 )$$

The third loss term LS3 (as shown in a block B83) focuses on “signal accuracy of complementary features”. As shown by arrows A85 and A86, the third loss term LS3 constrains that features generated by the adapter must possess physiological significance.

Taking an estimated physiological signal EBS_CF1 corresponding to the first complementary feature CF1 as an example, a loss function

$${ℒ}_{A}^{R}$$

thereof is defined as:

$${ℒ}_{A}^{R} = \frac{1}{\vert {\hat{S}}^{R} \vert} {\sum }_{{\hat{s}}_{i , j}^{R} ∈ {\hat{S}}^{R}} NP ( {\hat{s}}_{i , j}^{R} , s ) \qquad ( 9 )$$

wherein,

$${\hat{s}}_{i , j}^{R}$$

is a physiological signal estimated based on the first complementary feature. Similarly, a loss function

$${ℒ}_{A}^{N}$$

of the second complementary feature is defined as:

$${ℒ}_{A}^{N} = \frac{1}{\vert {\hat{S}}^{N} \vert} {\sum }_{{\hat{s}}_{i , j}^{N} ∈ {S}^{N}} NP ( {\hat{s}}_{i , j}^{N} , s ) \qquad ( 9 )$$

Finally, as shown in a table at a bottom right corner of FIG. 8, a total objective function TF( [symbol] ₜₒₜₐₗ) is a weighted sum of all the aforementioned loss terms:

$${ℒ}_{total} = {ℒ}_{np}^{All} + {ℒ}_{A}^{R} + {ℒ}_{A}^{N} + {ℒ}_{cos}^{R} + {ℒ}_{cos}^{N} \qquad ( 10 )$$

The training device calculates gradients according to the objective function TF, and performs Backpropagation as shown by an arrow A87. As shown in a block B84, this update process jointly adjusts parameters of the first/second feature extraction modules (211, 212), the first/second adapters (221, 222), and the reliability evaluation and signal estimation modules (230, 240), so as to minimize a total loss.

It should be noted that in another embodiment, as shown by a dashed arrow A88, a calculation scope of the first loss term LS1 may further extend to cover an “enhanced feature” level. Specifically, the signal estimation module 240 also estimates an enhanced signal set EBS' (including EBS_EF1 and EBS_EF2) based on the first enhanced feature EF1 and the second enhanced feature EF2 respectively. The training device or the processor 110 may further calculate a negative Pearson correlation coefficient between EBS_EF1 and the real physiological signal RBS, and a negative Pearson correlation coefficient between EBS_EF2 and RBS. Incorporating these intermediate-level losses into the first loss term LS1 for joint optimization may be regarded as a Deep Supervision mechanism, ensuring that the enhanced features after intra-modal weighting and cross-modal transformation possess high physiological signal accuracy before entering a final fusion stage, thereby further improving convergence speed and inference robustness of the model.

Referring to FIG. 9B, FIG. 9B further illustrates a training mechanism of the adapter module 220, showing how the first adapter 221 and the second adapter 222 are jointly optimized by the second loss term LS2 (structural constraint) and the third loss term LS3 (signal constraint).

In an embodiment, as shown in a block B94 (corresponding to an arrow A93-1), this is a training path of the first adapter 221. The first adapter 221 receives the second original feature RF2 (from the near-infrared modality) and converts it into a first complementary feature CF1 (used to simulate the visible light modality).

In order to ensure quality of CF1, the system applies dual constraints:

(1) Structural consistency constraint: calculating a cosine similarity loss (corresponding to

$${ℒ}_{cos}^{R}$$

labeled in the figure) between CF1 and the real first original feature RF1. This loss term enables the first adapter 221 to learn to map features to a feature space distribution same as that of RF1.

(2) Signal accuracy constraint: inputting CF1 into an estimator E_{R} corresponding to the first modality in the signal estimation module 240 so as to generate an estimated physiological signal, and calculating a negative Pearson correlation loss (corresponding to

$${ℒ}_{A}^{R}$$

labeled in the figure) between it and the real physiological signal RBS. This loss term requires CF1 to preserve decodable physiological information.

Similarly, as shown in a block B95 (corresponding to an arrow A93-2), this is a training path of the second adapter 222. The second adapter 222 receives the first original feature RF1 (from the visible light modality) and converts it into a second complementary feature CF2 (used to simulate the near-infrared modality). The system similarly applies dual constraints:

(1) Structural consistency constraint: calculating a cosine similarity loss (corresponding to

$${ℒ}_{cos}^{N}$$

labeled in the figure) between CF2 and the real second original feature RF2.

(2) Signal accuracy constraint: inputting CF2 into an estimator E_{N} corresponding to the second modality, and calculating a negative Pearson correlation loss (corresponding to

$${ℒ}_{A}^{N}$$

labeled in the figure) between its estimated physiological signal and RBS.

In other words, through joint constraints of the aforementioned dual losses, the adapter module 220 achieves a transformation effect of “taking care of both internal and external aspects”. Wherein, the structural loss ( [symbol] _{cos}) ensures that the transformed feature “looks like” the target modality in terms of numerical distribution and feature space (e.g., forcing the pseudo RGB feature to possess mathematical characteristics of a real RGB feature, making it compatible with an estimator trained for RGB); while the signal loss ( [symbol] _{A}) ensures that the feature “actually” carries correct physiological information in content (i.e., ensuring that the feature can restore a waveform consistent with a real heart rate after decoding). This mechanism avoids the adapter generating only meaningless noise, or generating features that are meaningful but have inconsistent formats.

Specifically, the first adapter 221 enables the near-infrared feature to possess a structural form of the visible light feature, so as to be interpreted by the estimator E_{R} trained for visible light; conversely, the second adapter 222 enables the visible light feature to possess a structural form of the near-infrared feature. This bidirectional and dual-constraint training strategy ensures that the complementary features can effectively exert an effect of “complementing each other's strengths and weaknesses” in a subsequent fusion stage, for example, allowing the visible light modality to obtain an ability to resist motion interference, or allowing the near-infrared modality to obtain an ability to resist light change.

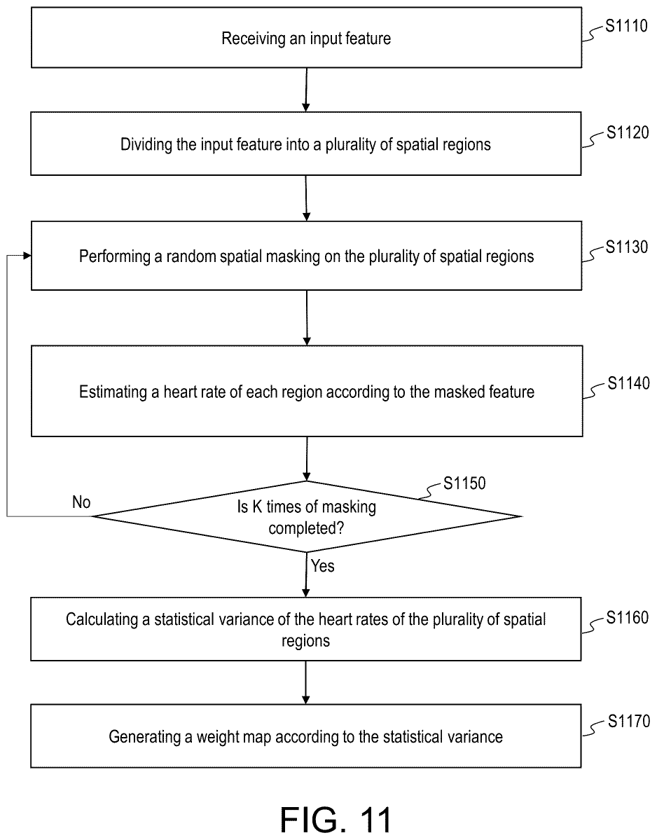

Referring to FIG. 11, in an embodiment, the reliability evaluation mechanism executed by the reliability evaluation and feature fusion module 230 comprises steps S1110 to S1170. A core purpose of this mechanism lies in quantifying spatial uncertainty of features, and generating a weight map accordingly, so as to suppress influence of unreliable regions in a subsequent fusion process.

In step S1110, the module receives an input feature. The input feature may be an original feature (RF1, RF2), a complementary feature (CF1, CF2), or an enhanced feature (EF1, EF2), depending on whether it is currently in an intra-modal fusion stage or a cross-modal fusion stage.

In step S1120, the module divides the input feature into a plurality of spatial regions (e.g., a q×q grid) in a spatial dimension. Each region corresponds to a specific anatomical location of the face of the subject (such as a forehead, a cheek).

In step S1130, the module performs a random spatial masking on the plurality of spatial regions. Specifically, the module randomly selects partial regions and sets feature values thereof to zero or suppresses them (similar to a Dropout mechanism). This step aims to simulate a scenario of partial feature loss or interference, so as to test robustness of the feature of the region for physiological signal estimation.

In step S1140, the module estimates a heart rate of each region according to the masked feature. Specifically, the masked feature is sent into the signal estimation module 240, decoded into a temporal rPPG signal, and a corresponding heart rate value (HR) is calculated via spectrum analysis.

In step S1150, the system determines whether K masking tests have been completed. If not, the flow returns to step S1130 to repeat execution with a different masking pattern; if yes, the flow proceeds to step S1160. Through this repeated perturbation process, the system can obtain K heart rate estimation values for each spatial region (i,j) under K different masking conditions.

In step S1160, the module calculates a statistical variance of the heart rate of each spatial region. The variance v_{i,j} may be defined as:

$${v}_{i , j} = \frac{1}{K} {\sum }_{k = 1}^{K} {( {HR}_{i , j}^{k} - μ )}^{2} \qquad ( 11 )$$

wherein

$${HR}_{i , j}^{k}$$

is an estimated heart rate after a k-th masking, and u is an average heart rate. The variance v_{i,j} directly reflects prediction uncertainty of the region: a larger variance represents that the region (possibly due to shadow, hair occlusion, or motion artifacts) is extremely sensitive to perturbation, so its feature is unreliable; conversely, it represents that the feature is stable and reliable.

In step S1170, the module generates a weight map according to the statistical variance. When performing weighted fusion of two sets of features (e.g., feature 1 and feature 2), this embodiment adopts a weighting strategy of “relative reliability”. Taking a weight

$${w}_{i , j}^{1}$$

of feature 1 as an example, a calculation formula thereof is as follows:

$${w}_{i , j}^{1} = \frac{exp ( {v}_{i , j}^{2} )}{exp ( {v}_{i , j}^{1} ) + exp ( {v}_{i , j}^{2} )} \qquad ( 12 )$$

wherein

$${v}_{i , j}^{1} and {v}_{i , j}^{2}$$

are variances of feature 1 and feature 2 in the region respectively. This formula embodies a logic of “reverse weighting”: if the variance

$${v}_{i , j}^{2}$$

of feature 2 in a certain region is very large (i.e., feature 2 is unreliable), a numerator term

$$exp ( {v}_{i , j}^{2} )$$

becomes large, making feature 1 obtain a higher weight

$${w}_{i , j}^{1}$$

in the region, thereby allowing feature 1 to dominate information contribution of the region.

Through the above mechanism, the system can dynamically generate weight maps suitable for intra-modal fusion (WM1R, WMIC, etc.) and weight maps suitable for cross-modal fusion (WM1E, WM2E). In addition, in an inference stage, the present disclosure provides two implementation modes: (1) Dynamic mode: executing the aforementioned random masking and variance calculation of S1130-S1170 in real-time during inference, so as to dynamically adjust weights in response to current environment (such as driving light changes), which has the highest accuracy; (2) Static mode: directly applying fixed weight maps learned and stored in the training stage, which has lower computational cost and is suitable for resource-constrained embedded systems.

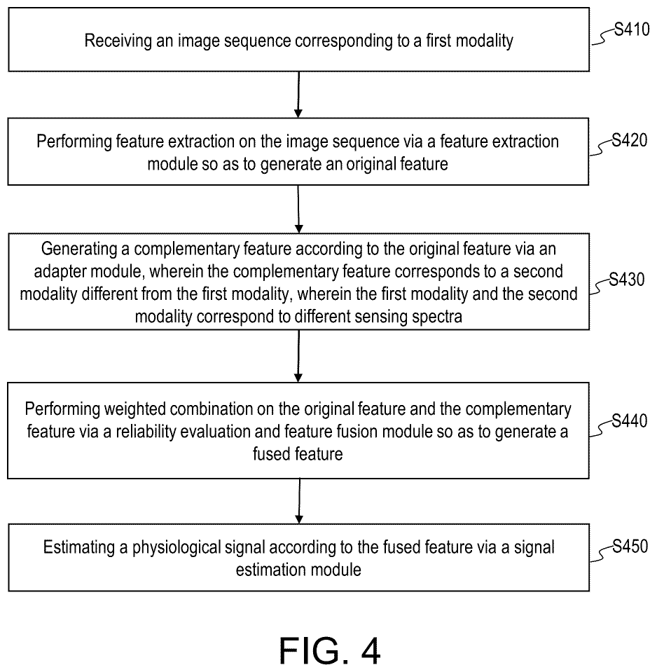

Referring to FIG. 4, in an embodiment, the physiological signal estimation method based on a single-modal source of the present disclosure comprises steps S410 to S450. This method is particularly suitable for a scenario where the physiological signal estimation system 100 is only configured with a single camera, or one of the cameras temporarily fails.

In step S410, the processor 110 receives an image sequence corresponding to a first modality. The first modality may depend on hardware configuration. For example, if the system is only configured with a visible light camera, a visible light image sequence is received; if only a near-infrared camera is configured, a near-infrared image sequence is received.

In step S420, the processor 110 performs feature extraction on the image sequence via a feature extraction module, so as to generate an original feature. Specifically, if the input is a visible light image, the first feature extraction module 211 extracts a first original feature RF1; if the input is a near-infrared image, the second feature extraction module 212 extracts a second original feature RF2.

In step S430, the processor 110 generates a complementary feature according to the original feature via an adapter module. The complementary feature corresponds to a second modality different from the first modality, and the first modality and the second modality correspond to different sensing spectra. This step embodies a core advantage of the present disclosure: utilizing a trained adapter to “simulate” information of a missing modality. For example, when the input is a visible light image (first modality), the processor 110 utilizes the second adapter 222 to convert RF1 into a second complementary feature CF2 simulating a near-infrared modality;

conversely, when the input is a near-infrared image, the first adapter 221 is utilized to convert RF2 into a first complementary feature CF1 simulating a visible light modality.

In step S440, the processor 110 performs weighted combination on the original feature and the complementary feature via the reliability evaluation and feature fusion module 230, so as to generate a fused feature. In this step, the module 230 may adopt a dynamic mode (calculating variance in real-time) or a static mode (applying pre-trained weights) to generate a weight map, and execute weighted fusion accordingly. Thereby, the system can effectively combine details of a real modality and complementary characteristics of a simulated modality (such as anti-light or anti-motion interference), improving robustness of features.

In step S450, the processor 110 estimates a physiological signal according to the fused feature via the signal estimation module 240. Specifically, the signal estimation module 240 decodes the fused feature into a temporal rPPG signal, and determines a heart rate value via spectrum analysis (such as calculating PSD). In a further embodiment, the processor 110 may capture a plurality of time segments (e.g., every 10 seconds, with overlapping intervals) from the image sequence, and perform statistical processing (such as calculating an average or a median) after estimating physiological signals respectively, so as to generate a more stable final physiological signal output.

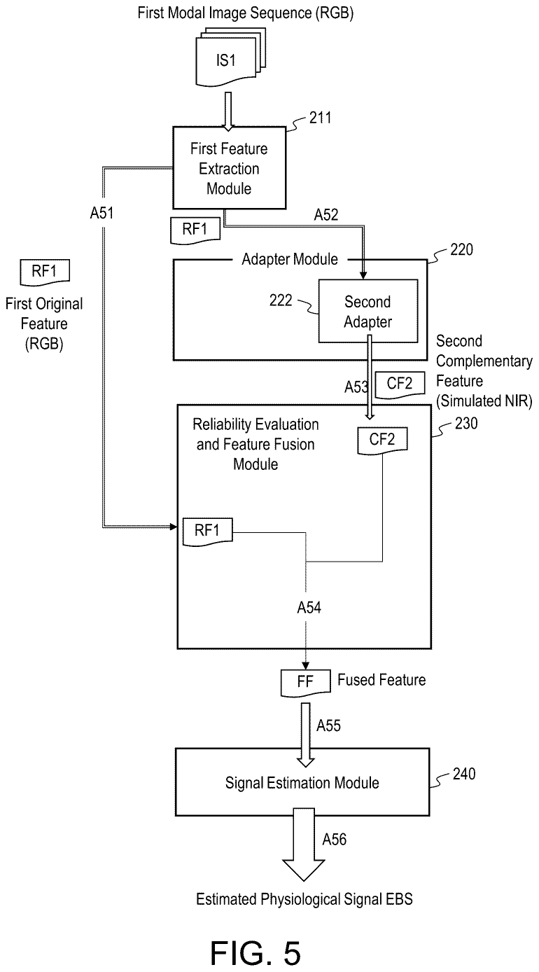

Referring to FIG. 5, a detailed architecture and data flow of the physiological signal estimation method based on a single-modal source are shown. This embodiment takes a scenario where an input is a first modality (e.g., visible light/RGB modality) as an example for description.

First, the first modal image sequence IS1 is input to the first feature extraction module 211. The first feature extraction module 211 performs convolutional operations on the image sequence so as to extract spatiotemporal features, so as to generate a first original feature RF1. As shown by arrows A51 and A52, the first original feature RF1 is directly transmitted to a fusion module at a back end on one hand, and is diverted to the adapter module 220 on the other hand.

Next, the adapter module 220 utilizes the second adapter 222 therein to perform cross-modal transformation. As shown by an arrow A53, the second adapter 222 receives the first original feature RF1 and converts it into a second complementary feature CF2. A purpose of the second complementary feature CF2 lies in simulating feature performance of a second modality (e.g., near-infrared/NIR modality). Specifically, since the second adapter 222 has learned a mapping relationship from RGB to NIR in the training stage, the generated CF2 can possess a characteristic of the NIR modality being “less sensitive to ambient light changes” to a certain extent, so as to provide complementary information simulated as NIR for the original RGB feature.

Subsequently, the reliability evaluation and feature fusion module 230 receives the first original feature RF1 from a path A51 and the second complementary feature CF2 from a path A53. As shown by an arrow A54, the module 230 performs weighted combination on the two sets of features so as to generate a fused feature FF. In this step, the module 230 dynamically adjusts contribution ratios of RF1 and CF2 according to spatial reliability of the features (e.g., via the aforementioned weight map mechanism). For example, if strong reflection occurs in a certain region of the RGB image causing RF1 to be unreliable, the system automatically increases a weight of CF2 in the region, utilizing the simulated NIR feature so as to fill in information.

Finally, as shown by an arrow A55, the fused feature FF is sent into the signal estimation module 240. The signal estimation module 240 decodes the high-dimensional fused feature, and as shown by an arrow A56, outputs a final estimated physiological signal EBS (e.g., a heart rate value).

It is worth noting that the architecture shown in FIG. 5 is also applicable to a scenario where an input is a second modality (e.g., a near-infrared modality). In this case, the input becomes IS2, the feature extraction module becomes the second feature extraction module 212, the adapter becomes the first adapter 221 (used so as to generate a first complementary feature CF1 simulating RGB), and the rest of the fusion and estimation processes can be deduced by analogy. This architecture design ensures that the system can maintain relatively accurate physiological signal estimation via a “self-simulation compensation” mechanism regardless of which single-modal input is used.

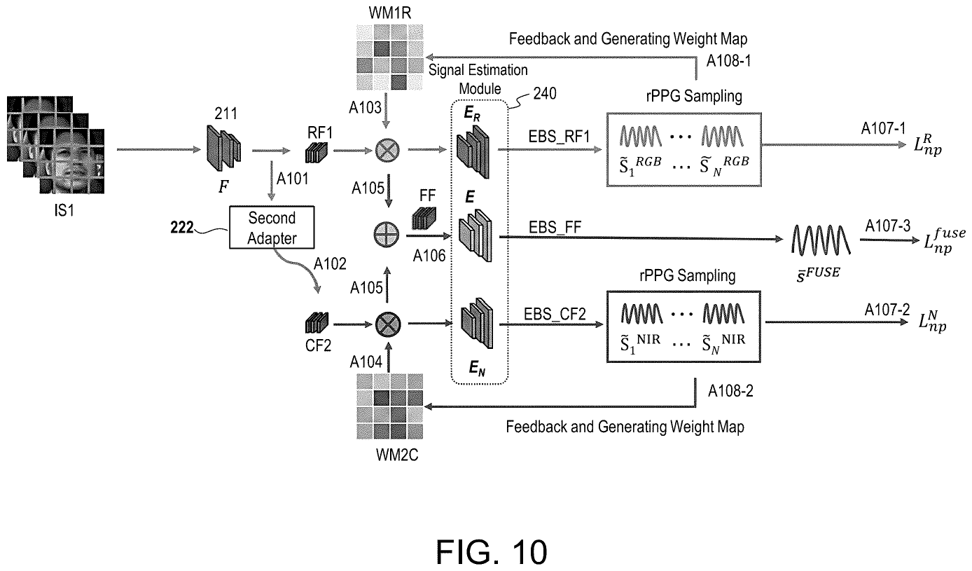

Referring to FIG. 10, in an embodiment, FIG. 10 illustrates in detail an operation schematic diagram of the multi-modal physiological signal estimation model based on a single-modal image sequence in an inference stage, particularly showing a feedback generation mechanism of a weight map. This embodiment takes a scenario where an input is a first modality (e.g., visible light/RGB modality) as an example for description.

First, the first modal image sequence IS1 is input to the first feature extraction module 211 (labeled as F). As shown by an arrow A101, the first feature extraction module 211 extracts a first original feature RF1. Next, as shown by an arrow A102, the second adapter 222 receives the first original feature RF1 and converts it into a second complementary feature CF2 (used so as to simulate a second modality/NIR modality).

Subsequently, the system performs reliability evaluation and feature fusion. As shown by an arrow A103, the first original feature RF1 is element-wise multiplied (®) by a first weight map WM1R; as shown by an arrow A104, the second complementary feature CF2 is element-wise multiplied by a second weight map WM2C. Next, as shown by an arrow A105, the two sets of weighted features are added (⊕) so as to generate a fused feature FF.

The signal estimation module 240 is responsible for decoding each feature into a physiological signal. Specifically, the signal estimation module 240 comprises three estimators: (1) a first estimator E_{R}: receiving the weighted first original feature RF1 and outputting a first estimated physiological signal EBS_RF1 (labeled as \tilde{S}^{RGB}); (2) a second estimator E_{N}: receiving the weighted second complementary feature CF2 and outputting a second estimated physiological signal EBS_CF2 (labeled as \tilde{S}^{NIR}); (3) a fusion estimator E: as shown by an arrow A106, receiving the fused feature FF and outputting a fused estimated physiological signal EBS_FF (labeled as s̅^{FUSE}).

In an embodiment, when the physiological signal estimation system 100 is configured with a contact physiological sensor (such as a steering wheel heart rate sensor), the processor 110 may obtain a real physiological signal RBS as a supervisory label. Estimated signals generated by each estimator of the signal estimation module 240 may also be used for calculating loss terms so as to perform retraining. As shown by an arrow A107-1, after the estimated physiological signal EBS_RF1 based on the first original feature RF1 is sampled via rPPG, a negative Pearson correlation loss

$${L}_{n p}^{R}$$

between it and the real physiological signal is calculated. As shown by an arrow A107-2, after the estimated physiological signal EBS_CF2 based on the second complementary feature CF2 is sampled via rPPG, a negative Pearson correlation loss

$${L}_{np}^{N}$$

between it and the real physiological signal is calculated. As shown by an arrow A107-3, a negative Pearson correlation loss

$${L}_{np}^{fuse}$$

between the estimated physiological signal EBS_FF based on the fused feature FF and the real physiological signal is calculated. The training device jointly optimizes model parameters according to the aforementioned loss terms by constraining these loss terms.

It is worth noting that FIG. 10 also reveals a generation mechanism of the weight map. As shown by arrows A108-1 and A108-2, outputs of the estimators E_{R} and E_{N} pass through an rPPG sampling procedure (rPPG Sampling, e.g., generating a plurality of sets of predicted signals [symbol] via a Dropout mechanism). The system calculates a statistical variance of these sampled signals, and feeds back variance information so as to generate the aforementioned weight maps (WM1R, WM2C). A region with a large variance represents that a feature there is unreliable, and thus is assigned a lower weight in the weight map.

In another embodiment, the reliability evaluation mechanism adopted by the reliability evaluation and feature fusion module 230 may select a dynamic mode or a static mode according to application requirements. When the dynamic mode is adopted, the processor 110 executes random spatial masking in real-time during inference so as to generate the weight map. Specifically, after receiving the image sequence and generating the original feature and the complementary feature, the processor 110 executes steps S1110 to S1160 described in FIG. 11 in real-time, and dynamically generates the weight map according to current feature content. An advantage of the dynamic mode lies in that the weight map can reflect influence of current environmental conditions on reliability of each spatial region, which is suitable for application scenarios with large environmental changes, such as a scenario where light conditions change continuously during vehicle driving.

When the static mode is adopted, the processor 110 applies a weight map pre-calculated in a training stage. Specifically, the training device executes the reliability evaluation mechanism described in FIG. 11 on a training dataset in the training stage, calculates an average reliability of each spatial region, and generates a preset weight map accordingly. The preset weight map is stored in the storage device 120, and the processor 110 directly loads the preset weight map during inference so as to perform feature weighted combination, without executing random spatial masking and heart rate variance calculation in real-time. An advantage of the static mode lies in that it can reduce computational burden during inference, which is suitable for application scenarios where the environment is relatively stable or computational resources are limited.

In another embodiment, the processor 110 may automatically switch between the dynamic mode and the static mode according to environmental conditions. When the processor 110 detects that ambient light or a state of the subject changes, the processor 110 switches to the dynamic mode so as to respond to environmental changes; when the environmental conditions tend to be stable, the processor 110 switches to the static mode so as to save computational resources.

Summarizing descriptions of FIG. 4, FIG. 5, and FIG. 10, the physiological signal estimation method based on a single-modal source of the present disclosure enables the physiological signal estimation system 100 to still generate a complementary feature corresponding to another modality via the adapter module 220 in a case where only a single-modal camera is provided, and perform weighted combination in combination with the reliability evaluation and feature fusion module 230, so as to estimate a physiological signal. When the input is a visible light image sequence, the second adapter 222 generates a second complementary feature CF2 simulating a near-infrared modality; when the input is a near-infrared image sequence, the first adapter 221 generates a first complementary feature CF1 simulating a visible light modality. The reliability evaluation mechanism may adopt the dynamic mode or the static mode. The dynamic mode generates the weight map in real-time during inference so as to respond to environmental changes, and the static mode applies the pre-calculated weight map so as to reduce computational burden. Through the above technical solution, the physiological signal estimation system 100 of the present disclosure can operate under different hardware configurations, improving flexibility of system deployment.

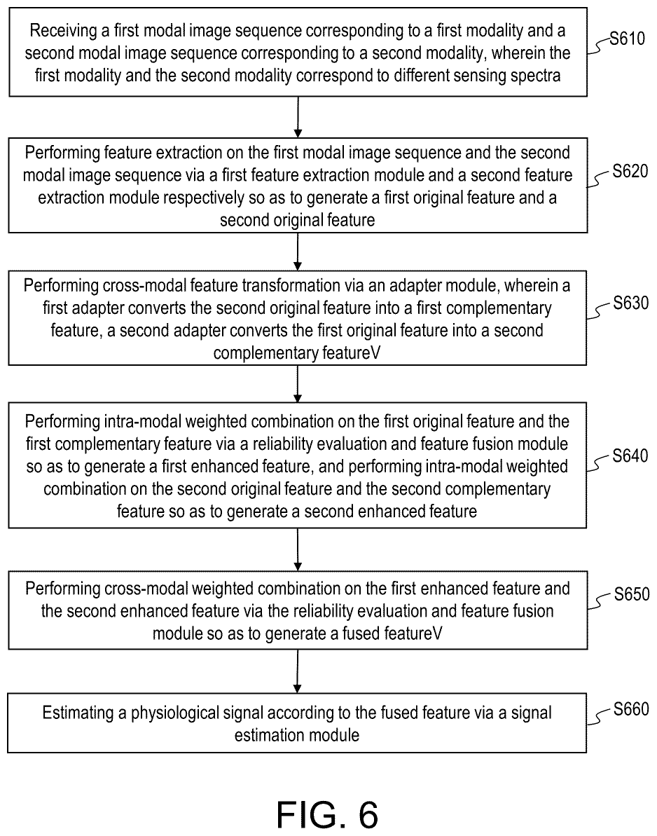

Referring to FIG. 6, in an embodiment, the physiological signal estimation method based on a dual-modal source of the present disclosure comprises steps S610 to S660. This method is suitable for a scenario where the physiological signal estimation system 100 is simultaneously configured with the first camera 141 (such as RGB) and the second camera 142 (such as NIR), aiming to achieve more accurate physiological signal estimation through deep fusion of dual-modal information.

In step S610, the processor 110 receives a first modal image sequence IS1 corresponding to a first modality and a second modal image sequence IS2 corresponding to a second modality. Wherein, the first modality and the second modality correspond to different sensing spectra. In order to ensure spatial and temporal consistency of subsequent feature fusion, the two sets of image sequences are Synchronized Capture, and field of views thereof cover the same facial area of the subject (see FIG. 1).

In step S620, the processor 110 performs feature extraction on the image sequences via a first feature extraction module and a second feature extraction module respectively. Specifically, the first feature extraction module 211 extracts a first original feature RF1 from IS1, and the second feature extraction module 212 extracts a second original feature RF2 from IS2. This step may utilize a multi-core or GPU architecture of the processor 110 to perform parallel computing, so as to improve processing efficiency.

In step S630, the processor 110 performs cross-modal feature transformation via an adapter module. This step executes bidirectional transformation: the first adapter 221 converts RF2 into a first complementary feature CF1 simulating the first modality; the second adapter 222 converts RF1 into a second complementary feature CF2 simulating the second modality. This mechanism lays a foundation for subsequent cross-enhancement.

In step S640, the processor 110 executes intra-modal weighted combination via the reliability evaluation and feature fusion module so as to generate enhanced features. Specifically, the system fuses RF1 and CF1 so as to generate a first enhanced feature EF1, and fuses RF2 and CF2 so as to generate a second enhanced feature EF2. A technical effect of this step lies in “Cross-Enhancement”: EF1 not only contains original information of RGB, but also fuses anti-motion interference characteristics from NIR; EF2 not only contains original information of NIR, but also fuses anti-light change characteristics from RGB. The fusion process is weighted based on weight maps (such as WM1R, WMIC, etc.) generated by the reliability evaluation mechanism, so as to suppress unreliable regions.

In step S650, the processor 110 performs cross-modal weighted combination on the first enhanced feature EF1 and the second enhanced feature EF2 via the reliability evaluation and feature fusion module, so as to generate a fused feature FF. In this stage, the system evaluates spatial reliability of EF1 and EF2 again and generates corresponding weight maps (such as WM1E, WM2E), ensuring that the final fused feature FF gathers the best information fragments of dual modalities.

In step S660, the processor 110 estimates a physiological signal according to the fused feature FF via the signal estimation module. The signal estimation module decodes the fused feature FF into a temporal signal and calculates a heart rate. Since the fused feature FF has undergone two stages of reliability weighting and cross-enhancement, physiological information contained therein is purer and more robust compared to single-modal features, and can effectively resist various types of interference in complex environments.

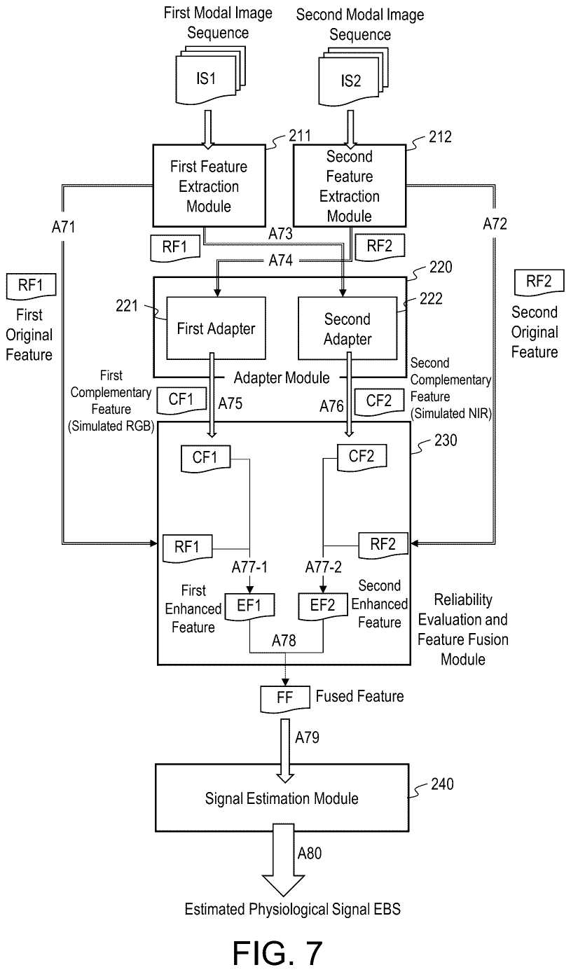

Referring to FIG. 7, in an embodiment, FIG. 7 shows a detailed architecture and data flow of the physiological signal estimation method based on a dual-modal source. This architecture is suitable for a scenario where the physiological signal estimation system 100 is simultaneously equipped with the first camera 141 and the second camera 142, aiming to achieve more reliable physiological signal estimation through deep fusion of dual-modal information.

First, the first modal image sequence IS1 and the second modal image sequence IS2 are respectively input to corresponding feature extraction modules. As shown by an arrow A71, the first feature extraction module 211 performs feature extraction on IS1, generating a first original feature RF1. As shown by an arrow A72, the second feature extraction module 212 performs feature extraction on IS2, generating a second original feature RF2.

Next, the adapter module 220 executes bidirectional cross-modal transformation. As shown by an arrow A74, the first adapter 221 receives RF2 and converts it into a first complementary feature CF1 (simulating the first modality). As shown by an arrow A73, the second adapter 222 receives RF1 and converts it into a second complementary feature CF2 (simulating the second modality). This step provides necessary complementary information for subsequent cross-enhancement.

Subsequently, the reliability evaluation and feature fusion module 230 executes two-stage feature fusion. The first stage is “intra-modal weighted combination”. As shown by arrows A71 and A75, the reliability evaluation and feature fusion module 230 receives RF1 and CF1, and performs weighted fusion utilizing corresponding weight maps (WM1R, WM1C), as shown by an arrow A77-1, so as to generate a first enhanced feature EF1. Similarly, as shown by arrows A72 and A76, the module 230 receives RF2 and CF2, and performs weighted fusion utilizing corresponding weight maps (WM2R, WM2C), as shown by an arrow A77-2, so as to generate a second enhanced feature EF2. A purpose of this stage lies in utilizing complementary features so as to repair information in original features damaged by environmental interference (such as light or motion).

The second stage is “cross-modal weighted combination”. As shown by an arrow A78, the module 230 further receives EF1 and EF2, and performs weighted fusion on the two sets of enhanced features utilizing cross-modal weight maps (WM1E, WM2E), so as to generate a final fused feature FF. In this step, the system evaluates spatial reliability of EF1 and EF2 again, ensuring that the final fused feature FF gathers the best information fragments of dual modalities.

Finally, as shown by an arrow A79, the fused feature FF is sent into the signal estimation module 240. The signal estimation module 240 decodes the high-dimensional fused feature, and as shown by an arrow A80, outputs a final estimated physiological signal EBS (e.g., a heart rate value).

Regarding technical implications of the two-stage fusion mechanism, taking a vehicle scenario as an example: when a vehicle enters or exits a tunnel causing drastic light changes, RF1 of the visible light modality may be damaged, but CF1 (converted via the adapter) from the near-infrared modality can provide stable information, repairing EF1 through the first stage fusion. If the driver rotates the head simultaneously causing motion artifacts in RF2 of the near-infrared modality, CF2 from the visible light modality can provide stable information, repairing EF2. Finally, the second stage fusion combines EF1 and EF2, ensuring that the system can output a stable physiological signal regardless of light changes or motion interference.

Regarding the two-stage fusion mechanism adopted by the reliability evaluation and feature fusion module 230, its core technical implication lies in achieving stronger robustness through “intra-modal self-compensation enhancement” and “cross-modal defect compensation”.

More specifically, in the “intra-modal weighted combination” of the first stage, the system aims to solve innate defects of a single modality. Taking the vehicle scenario as an example, when the vehicle passes through an environment with drastic light changes (such as entering/exiting a tunnel or passing through bridge shadows), image quality captured by the first camera 141 (visible light modality) may drop instantly, causing high variance (i.e., unreliability) in partial spatial regions of the first original feature RF1. At this time, the first complementary feature CF1 converted from the near-infrared modality by the first adapter 221 can provide relatively stable feature information because it inherits the characteristic of “resistance to light changes” of near-infrared. Through the weight maps (WM1R, WMIC) generated by the reliability evaluation mechanism, the system automatically reduces a weight of RF1 and increases a weight of CF1, so that the generated first enhanced feature EF1 can repair defects caused by light and shadow while preserving RGB details.

Conversely, when the driver performs actions such as rapid head rotation or speaking, the second camera 142 (near-infrared modality) is prone to generating Motion Artifacts, causing the second original feature RF2 to be unreliable. At this time, the second complementary feature CF2 converted from the visible light modality by the second adapter 222 can provide clear structural information because it inherits the characteristic of “resistance to motion interference” of the visible light modality. The system adjusts weights (WM2R, WM2C) accordingly, utilizing CF2 so as to correct RF2, so as to generate a stable second enhanced feature EF2.

Next, in the “cross-modal weighted combination” of the second stage, the system further integrates the first enhanced feature EF1 and the second enhanced feature EF2 that have completed self-repair. Since EF1 and EF2 represent optimized visible light and near-infrared features respectively, there may still be overall differences in pros and cons between the two in different environments (e.g., in an extremely dark environment, even the repaired EF1 is not as effective as EF2). Therefore, the system utilizes cross-modal weight maps (WM1E, WM2E) again so as to dynamically evaluate reliability of both, and performs weighted fusion on the two so as to generate the final fused feature FF.

Simply put, the dual-modal inference method of the present disclosure achieves multi-level noise protection through the aforementioned two-stage mechanism. The first stage utilizes “complementary features” so as to solve specific weaknesses (light/shadow or motion) of a single modality, and the second stage utilizes “enhanced features” for strong alliance. In conjunction with the dynamic weight mechanism based on random spatial masking and variance calculation, the system can adaptively respond to complex light and motion changes in the driving environment, ensuring continuity and precision of physiological signal estimation.

In an experimental example, the multi-modal physiological signal estimation model of the present disclosure is evaluated on a plurality of public datasets. The datasets comprise synchronized visible light and near-infrared facial image sequences captured in controlled indoor environments and actual driving scenarios. Experimental conditions cover various scenarios such as static state, slight motion, and light changes.

Experimental results show that in a scenario of single-modal input, the model configured with the adapter module achieves lower Mean Absolute Error (MAE) and Root Mean Square Error (RMSE) under various test conditions compared to a model without the adapter module. Taking a slight motion condition in a driving scenario as an example, the MAE of the visible light single-modal model configured with the adapter module is reduced from 12.04 bpm to 8.63 bpm.

In a scenario of dual-modal input, a feature fusion strategy adopting the reliability evaluation mechanism achieves better estimation results under various test conditions compared to a direct feature fusion strategy. Taking the slight motion condition in the driving scenario as an example, the MAE of the dual-modal model adopting the reliability evaluation mechanism is 6.62 bpm, which is improved compared to 7.26 bpm of the direct fusion strategy.

In addition, estimation results of dual-modal fusion are superior to estimation results of single-modal configuration with the adapter module. Taking the slight motion condition in the driving scenario as an example, the MAE of the dual-modal fusion model is 6.62 bpm, which is better than 8.63 bpm of the visible light single-modal configuration with the adapter module and 9.59 bpm of the near-infrared single-modal configuration with the adapter module. This result shows that when image sequences of both modalities are available, integrating real features and complementary features of dual modalities via the reliability evaluation mechanism can obtain more accurate physiological signal estimation than single-modal simulation fusion.

The above experimental results verify effectiveness of the cross-modal feature transformation mechanism, the reliability evaluation and feature fusion mechanism, and the adapter module proposed by the present disclosure in improving accuracy of physiological signal estimation.

In summary, the training method for a multi-modal physiological signal estimation model, the physiological signal estimation method, and the system provided by the present disclosure cope with differences between different sensing spectra (such as visible light and near-infrared) through the cross-modal feature transformation mechanism of the adapter module. By jointly optimizing the objective function containing signal accuracy and structural consistency, the model can realize complementary learning at the feature level, enabling a single modality to possess advantageous characteristics of another modality (e.g., the visible light modality obtains anti-motion interference ability, and the near-infrared modality obtains anti-light change ability), thereby effectively compensating for innate weaknesses of each modality.

In addition, combined with the reliability evaluation mechanism based on random spatial masking and statistical variance calculation, the present disclosure can generate a weight map so as to dynamically suppress feature contributions of unreliable regions (such as shadows or occlusions) and preserve high-confidence information. This mechanism not only achieves extreme robustness through two-stage fusion under dual-modal input, but more critically, effectively compensates for information of a missing modality utilizing complementary features under a single-modal input scenario, solving hardware failure or cost limitation problems. In conjunction with flexible switching between dynamic and static weight modes, the technical solution of the present disclosure can be widely adapted to complex and changeable application scenarios such as vehicle monitoring and telemedicine, improving practicality and accuracy of non-contact physiological signal estimation.

Furthermore, the technical solution of the present disclosure improves functionality of the image sensing system itself, rather than merely using sensors as data collection tools followed by post-processing of data. Through cross-modal feature transformation of the adapter module and dynamic weight adjustment of the reliability evaluation mechanism, the system can compensate for physical limitations caused by sensing spectrum characteristics of a single sensor at a feature level in real-time. Such improvement is directly targeted at signal processing capabilities of the sensor system itself, enabling it to output physiological signal estimation results that remain stable under various environmental interferences, rather than merely analyzing or classifying information content being processed.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A training method for a multi-modal physiological signal estimation model, comprising:
receiving a first modal image sequence corresponding to a first modality, a second modal image sequence corresponding to a second modality, and a real physiological signal corresponding to the first modal image sequence and the second modal image sequence, wherein the first modality and the second modality correspond to different sensing spectra;
performing feature extraction on the first modal image sequence via a first feature extraction module, so as to generate a first original feature;
performing feature extraction on the second modal image sequence via a second feature extraction module, so as to generate a second original feature;
performing cross-modal feature transformation via an adapter module, wherein the adapter module comprises a first adapter and a second adapter, the first adapter converts the second original feature into a first complementary feature, and the second adapter converts the first original feature into a second complementary feature; and
adjusting parameters of the multi-modal physiological signal estimation model so as to optimize an objective function, wherein the objective function is calculated based on the real physiological signal.

2. The training method of claim 1, wherein the objective function comprises a first loss term, a second loss term, and a third loss term.

3. The training method of claim 2, wherein the first loss term is used to constrain a similarity between the real physiological signal and physiological signals estimated based on the first modality and the second modality.

4. The training method of claim 2, wherein the second loss term is used to constrain a structural similarity between the first complementary feature and the first original feature, and constraints constrain a structural similarity between the second complementary feature and the second original feature.

5. The training method of claim 2, wherein the third loss term is used to constrain a similarity between the real physiological signal and physiological signals estimated by based on the first complementary feature and the second complementary feature.

6. The training method of claim 1, wherein the first modal image sequence is a visible light image sequence, and the second modal image sequence is a near-infrared image sequence.

7. The training method of claim 1, further comprising:
performing intra-modal weighted combination on the first original feature and the first complementary feature, so as to generate a first enhanced feature;
performing intra-modal weighted combination on the second original feature and the second complementary feature, so as to generate a second enhanced feature; and
performing cross-modal weighted combination on the first enhanced feature and the second enhanced feature, so as to generate a fused feature.

8. The training method of claim 7, wherein the intra-modal weighted combination and the cross-modal weighted combination are weighted based on a weight map generated by a reliability evaluation mechanism.

9. The training method of claim 8, wherein the reliability evaluation mechanism comprises:
dividing a feature into a plurality of spatial regions;
performing random spatial masking on the plurality of spatial regions;
estimating a heart rate of each region according to the masked feature; and
calculating a statistical variance of the heart rates of the plurality of spatial regions, so as to generate the weight map.

10. A physiological signal estimation method based on a single-modal source, adapted for a multi-modal physiological signal estimation model, the multi-modal physiological signal estimation model comprising a feature extraction module, an adapter module, a reliability evaluation and feature fusion module, and a signal estimation module, the method comprising:
receiving an image sequence corresponding to a first modality;
performing feature extraction on the image sequence via the feature extraction module, so as to generate an original feature;
generating a complementary feature according to the original feature via the adapter module, wherein the complementary feature corresponds to a second modality different from the first modality, wherein the first modality and the second modality correspond to different sensing spectra;
performing weighted combination on the original feature and the complementary feature via the reliability evaluation and feature fusion module, so as to generate a fused feature; and
estimating a physiological signal according to the fused feature via the signal estimation module.

11. The physiological signal estimation method based on a single-modal source of claim 10, wherein:
when the image sequence is a visible light image sequence, the complementary feature is used to simulate a feature of a near-infrared modality; and
when the image sequence is a near-infrared image sequence, the complementary feature is used to simulate a feature of a visible light modality.

12. The physiological signal estimation method based on a single-modal source of claim 10, wherein the reliability evaluation and feature fusion module generates a weight map based on a reliability evaluation mechanism, and performs weighted combination on the original feature and the complementary feature according to the weight map.

13. The physiological signal estimation method based on a single-modal source of claim 12, wherein the reliability evaluation mechanism adopts a dynamic mode, and performs random spatial masking in real-time during inference so as to generate the weight map.

14. The physiological signal estimation method based on a single-modal source of claim 12, wherein the reliability evaluation mechanism adopts a static mode, and applies a weight map pre-calculated during a training stage.

15. A physiological signal estimation method based on a dual-modal source, adapted for a multi-modal physiological signal estimation model, the multi-modal physiological signal estimation model comprising a first feature extraction module, a second feature extraction module, an adapter module, a reliability evaluation and feature fusion module, and a signal estimation module, the method comprising:
receiving a first modal image sequence corresponding to a first modality and a second modal image sequence corresponding to a second modality, wherein the first modality and the second modality correspond to different sensing spectra;
performing feature extraction on the first modal image sequence and the second modal image sequence via the first feature extraction module and the second feature extraction module respectively, so as to generate a first original feature and a second original feature;
performing cross-modal feature transformation via the adapter module, wherein a first adapter converts the second original feature into a first complementary feature, and a second adapter converts the first original feature into a second complementary feature;
performing intra-modal weighted combination on the first original feature and the first complementary feature via the reliability evaluation and feature fusion module so as to generate a first enhanced feature, and performing intra-modal weighted combination on the second original feature and the second complementary feature so as to generate a second enhanced feature;
performing cross-modal weighted combination on the first enhanced feature and the second enhanced feature, so as to generate a fused feature; and
estimating a physiological signal according to the fused feature via the signal estimation module.

16. The physiological signal estimation method based on a dual-modal source of claim 15, wherein the first modal image sequence is a visible light image sequence, and the second modal image sequence is a near-infrared image sequence.

17. The physiological signal estimation method based on a dual-modal source of claim 15, wherein the intra-modal weighted combination and the cross-modal weighted combination are weighted based on a weight map generated by a reliability evaluation mechanism.

18. A physiological signal estimation system, comprising:
an image capturing device, configured to capture an image sequence of at least one modality;
a processor, coupled to the image capturing device; and
a storage device, coupled to the processor, configured to store a multi-modal physiological signal estimation model, wherein the model comprises a plurality of code modules, and the plurality of code modules, after being executed by the processor, cause the processor to perform the following steps:
utilizing a first feature extraction module to perform feature extraction on a first modal image sequence so as to generate a first original feature;
utilizing a second adapter of an adapter module to convert the first original feature into a second complementary feature, wherein the second complementary feature corresponds to a second modality different from the first modality;
utilizing a reliability evaluation and feature fusion module to perform weighted combination on the first original feature and the second complementary feature so as to generate a fused feature; and
utilizing a signal estimation module to estimate a physiological signal according to the fused feature.

19. The physiological signal estimation system of claim 18, wherein the image capturing device comprises a first camera configured to capture a visible light image sequence or a near-infrared image sequence; or, the image capturing device further comprises a second camera, such that the first camera captures the visible light image sequence and the second camera captures the near-infrared image sequence.

20. The physiological signal estimation system of claim 19, wherein when the image capturing device comprises the second camera, the processor is further configured to perform the following steps:
utilizing a second feature extraction module to perform feature extraction on the near-infrared image sequence so as to generate a second original feature; and
utilizing a first adapter of the adapter module to convert the second original feature into a first complementary feature;
wherein the reliability evaluation and feature fusion module performs intra-modal weighted combination on the first original feature and the first complementary feature so as to generate a first enhanced feature, performs intra-modal weighted combination on the second original feature and the second complementary feature so as to generate a second enhanced feature, and performs cross-modal weighted combination on the first enhanced feature and the second enhanced feature so as to generate the fused feature.
